(19) United States
(12) Patent Application Publication
(10) Pub. No.: US 2005/0113738 A1
(43) Pub. Date: May 26, 2005

(54) Iontophoresis electrode device

(75) Inventors: Yasushi Fuchita, Tokyo (JP); Tatsuya Ogawa, Tokyo (JP); Saori Takahashi, Tokyo (JP); Takuji Oikawa, Tokyo (JP); Shuji Sato, Kanagawa (JP); Naruhito Higo, Kannondai (JP); Hiroyuki Maeda, Saga (JP); Kenji Mori, Ibaraki (JP)

Correspondence Address: Frommer Lawrence & Haug, 745 Fifth Avenue, New York, NY, 10151, US

(73) Assignee: HISAMITSU PHARMACEUTICAL CO., INC

(21) Appl. No.: 10/500,537

(22) PCT Filed: Jan. 9, 2003
(86) PCT No.: PCT/JP03/00121

(30) Foreign Application Priority Data
Jan. 10, 2002 (JP) 2002-003949

(51) Int. Cl. A61N 1/30

(52) U.S. Cl. 604/20

(57) ABSTRACT

There is provided an electrode device for iontophoresis capable of retaining a drug in a gel state with a force just enough so as not to disturb the treatment. In order to retain the gel (70), a sheet member (50) composed of a nonwoven fabric is laminated on a base film (20) including an electrode layer (30) so as to be integrated with the base film (20). The sheet member (50) has a property for allowing the gel (70) to permeate therein. Owing to this feature, the gel (70) loaded on the sheet member (50) is retained thereon with at least a part of the gel (70) permeated therein.

### TECHNICAL FIELD OF THE INVENTION

This invention relates to an iontophoresis technique in which a voltage is impressed on a gel containing a drug to be introduced into the body of a patient in order to make the drug migrate into the patient's body, and more particularly to an electrode device capable of correctly retaining a gel containing a drug.

### BACKGROUND OF THE INVENTION

Iontophoresis is, in general, a treatment technique for putting a portion containing a drug on the skin (derma and mucous membrane) of a patient and impressing a voltage thereon to cause the drug to make ion-migration so that the drug is introduced into the patient's body through the skin. The portion containing a drug includes a place for storing the drug and an electrode layer. A voltage coming from an external power supply is impressed on the electrode layer. That is, the portion containing a drug functions as an electrode device. The official gazette of Japanese Patent Application Laid-Open No. 2000-316991 discloses the idea for making the electrode device disposable, while the external power supply is repeatedly used. As a disposable electrode device, this official gazette proposes one form of an arrangement which comprises a sheet base having a concave part shaped by forming, and an electrode layer including an electrode main body part located at the bottom of the concave part and a lead part extending outward of the concave part from the electrode main body part, a conductive layer containing a drug being disposed on the electrode layer in the concave part. The conductive layer comprises a porous material having a high rate of porosity such as nonwoven fabric and plastic foam, and a drug contained in the porous material.

Problem to be Solved by the Invention

Accordingly, it will be possible to prepare a drug in any desired state of liquid or gel. It should be noted, however, that although the conductive layer containing a drug is located in the concave part, it is merely loaded on the electrode layer. Since the concave part is placed up side down at the time of actual iontophoresis treatment, there is such a fear that the conductive layer in the concave part is accidentally fallen from the concave part. Moreover, when the handling is taken in account, the drug is preferably in the state of gel which has a shape retentive property rather than in the state of liquid which has a fluidability. However, it is a very difficult job to sufficiently permeate the gel into the thick conductive layer from the top to the bottom.

The present invention has been accomplished in view of the above points. It is, therefore, an object of the present invention to provide an iontophoresis electrode device capable of retaining a gel with a force just enough so as not to disturb the treatment in case a drug is used in a state of gel.

It is another object of the present invention to provide an electrode device capable of not only effectively retaining a gel containing a drug but also controlling the contact surface between the gel and the patient's skin.

Further objects of the present invention will become more manifest from the following description.

### DISCLOSURE OF THE INVENTION

The present invention is based on the premise that a drug is used in the form of a gel having a shape retainability, and the gel is retained with respect to an electrode device. In order to retain the gel, a specific sheet member is laminated on a base film including an electrode layer and integrated with the base film. The sheet member has a property for allowing the gel to permeate therein, so that the gel disposed on the sheet member can be retained with at least a part of the gel permeated in the sheet member. As the sheet member, a porous material having a high rate of porosity such as the above-mentioned nonwoven fabric and plastic foam in the conductive layer can widely be used. However, the sheet member used in this invention is, for example, about 0.05 to 1 mm in thickness and thin compared with the thickness of the conductive layer. Moreover, the conductive layer is employed for containing and storing a drug, while the sheet member is used for retaining a gel containing a drug so that the gel will not fallen down and not used for storing a drug. In this invention, usually, the gel containing a drug is permeated only at one part thereof on the side contacting the sheet member into the sheet member and the remaining part of the gel is located at the upper part of the sheet member, i.e., at the outside of the sheet member. A nonwoven fabric is particularly preferable as a material of the sheet member in view of a force for retaining the gel.

Moreover, in case the sheet member is laminated on the base film, the sheet member can be bonded to the base film by fusing or press-fitting the material itself of the sheet member thereto. Preferably, an adhesive agent having a conductive property is used for bonding the sheet member to the base film. By doing so, the electrode layer on the base film and the gel on the sheet member side can be effectively electrically conducted to each other.

As the base film itself, a member obtained by laminating a plastic film and a metal film can widely be used, as in the case with the official gazette of Japanese Patent Application Laid-Open No. 2000-316991. The base film may be provided at the region where the gel containing a drug is disposed with a concave part, or it may be used in the form of a flat sheet without being provided with a concave part. In order to somewhat deform the electrode device itself so that the electrode device can intimately be contacted with the patient's skin, it is preferable that the base film can easily be bent by hand and the bent state of the base film can be retained. In this respect, a layer construction is desirable in which the plastic film and metal film are set to be 10 to 200 μm in thickness as taught by the official gazette of Japanese Patent Application Laid-Open No. H11-54855, and which has a return characteristic for returning the bent state of the plastic film to its original state and a shape retaining force for retaining the bent state of the metal film. 1 of the thickness of the metal film per 2 of the thickness of the plastic film is the border. Preferably, the thickness of the metal film and that of the plastic film are made equal and they are set within a range of 40 to 80 μm. As material of the plastic, polyethylene terephthalate, polyimide, polyethylene, polypropylene, polyethylene naphthalate or the like can be used. On the other hand, as material of the metal film, aluminum, copper, zinc, silver, gold or lead, or alloy thereof can be used. Moreover, as the base film, a plastic film single layer can also be used. As its material, the above-mentioned plastic material such as polyethylene terephthalate, which was used for being laminated with the metal film, can be used. The thickness of the single layer plastic film is preferably 10 to 300 μm, more preferably 20 to 200 μm, and most preferably 35 to 100 μm. Of the plastic material, polyethylene terephthalate is particularly preferable.

The electrode layer on the base film includes a main body part corresponding to the region where the gel containing a drug is disposed, and a lead part extending from the main body part. This electrode layer is preferably formed by printing such as a screen printing and a gravure printing. As material of the electrode layer, various electrode materials can be used. In case the electrode layer is formed by printing, a conductive paste ink, for example, can be used. In order to prevent the part of the electrode layer from contacting directly the patient's skin, an insulative layer is preferably provided in such a way as to surround the main body part of the electrode layer and traverse above the lead part. This insulative layer can also be formed by printing.

In the preferred embodiments of the present invention, the base film and the sheet member as component elements of the electrode apparatus are integrated altogether, with the electrode layer sandwiched therebetween. Accordingly, both the base film and the sheet member are cooperated with each other to exhibit the function for protecting the electrode layer. When it is taken into account that the electrode device is deformed, this protecting function could be important for the electrode device.

### BRIEF DESCRIPTION OF DRAWINGS

FIG. 1 is a plan view of a first embodiment of the present invention.

FIG. 2 is a sectional view of the first embodiment.

FIG. 3 is a plan view of a second embodiment of the present invention.

FIG. 4 is a sectional view of the second embodiment.

FIG. 5 is a sectional view of a third embodiment.

FIG. 6 is a plan view of a fourth embodiment.

FIG. 7 is a sectional view taken along the line 7-7 of FIG. 6.

FIG. 8 is a sectional view, corresponding to FIG. 7, showing a fifth embodiment.

### BEST MODE FOR CARRYING OUT THE INVENTION

### FIRST EMBODIMENT

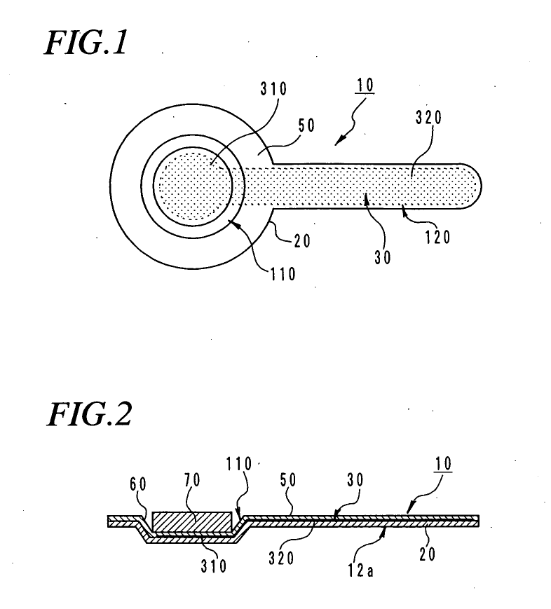

The first embodiment is an aluminum laminate cup type electrode device having a concave part. FIG. 1 shows its plan view, and FIG. 2 shows its sectional view. This electrode device 10 uses an aluminum laminate raw film obtained by laminating aluminum as material of the base film 20. The aluminum laminate raw film is a layer composing laminate film such as polyethylene terephthalate/aluminum/polyethylene terephthalate. Its thickness is 0.13 mm. A screen printing is made onto one surface of this aluminum laminate film using paste ink first, and then, the printed surface is dried at 130 degrees C. for two minutes, so that an electrode layer 30 is formed on that surface. This electrode layer 30 includes a circular main body part 310 having a diameter of about 17 to 18 mm, and a lead part 320 linearly extending from the main body part 310. The lead part is about 10 mm in width and about 35 mm in length.

Then, a conductive adhesive agent is coated onto the base film 20 including the electrode layer 30 by using a gravure plate cylinder, and a nonwoven fabric 50 as a sheet member is laminated on the coating layer. The nonwoven fabric 50 usable here is in such a wide range as having a thickness of about 0.05 to 1.0 mm, i.e., from a thin fabric to a thick fabric. The thickness of the nonwoven fabric 50 should be set to be smaller than the height of the gel 70 containing a drug. Particularly, it should be preferably set to be thin so as not impair such characteristics as bending and shape retainability of the base film 20. The laminating material obtained by laminating the nonwoven fabric 50, i.e., the laminating material having such a construction as the base film 20/the electrode layer 30/the nonwoven fabric 50 can be handled in the winding manner. In order to carry out an external electrical connection to the electrode layer 30, a local contact layer (not shown) is printed on the nonwoven fabric 50 covering the lead part 320 by using the conductive paste ink. As the conductive paste ink, for example, a silver paste is used for the anode and silver chloride paste is used for the cathode by taking electrolyte into account. As the external electrical connection method, other methods such as a method for removing a part of the nonwoven fabric 50 or a method for allowing an electrical contact means (for example, a clip) to pierce through the nonwoven fabric 50 without removing a part of the nonwoven fabric 50 may be employed.

The electrode layer 30 is, for example, about 15 μm in thickness. Since such thin electrode layer 30 is sandwiched between and protected by the base film 20 and the nonwoven fabric 50 which are thicker than at least the electrode layer 30, it has a sufficient resistance to bending, etc. Lastly, the laminating material having such a construction as the base film 20/the electrode layer 30/the nonwoven fabric 50 is subjected to sheet molding and punching work. By doing so, the electrode device 10 comprising a cup part 110 having a concave part 60 and a lead part 120 extending from the cup part 110 can be obtained. The concave part 60 of the cup part 110 has a depth of about 2 mm. When a gel 70 containing a drug is charged into the cup part 110, the gel 70 at the bottom of the concave part 60 which contacts the nonwoven fabric 50 is permeated into the nonwoven fabric 50 and retained by a large enough force so that the gel 50 will not be fallen down, even if, for example, the electrode device 10 is put upside down. The inside diameter of the concave part 60 of the cup part 110 is about 25 mm, and the outside diameter of the cup part 110 is about 40 mm.

The electrode device 10 comprises, in addition to the electrode layer 30, the nonwoven fabric 50 laid on the electrode layer 30, integral with the base film 20. The nonwoven fabric 50 has the same contour as the base film 20. The nonwoven fabric 50 effectively protects the electrode layer 30 which is mechanically not very strong, together with the base film 20. Moreover, the nonwoven fabric 50 integral with the base film 20 has such a function as to allow the gel 70 to permeate therein. The nonwoven fabric 50 retains the gel 70 with an appropriate retaining force with at least a part of the gel 70 permeated therein. In this electrode device 10, the electrode layer 30 is made electrically conductive with the gel 70 at the cup part 110 through a conductive adhesive agent and a part of the gel 70 permeated into the nonwoven fabric 50.

### SECOND EMBODIMENT

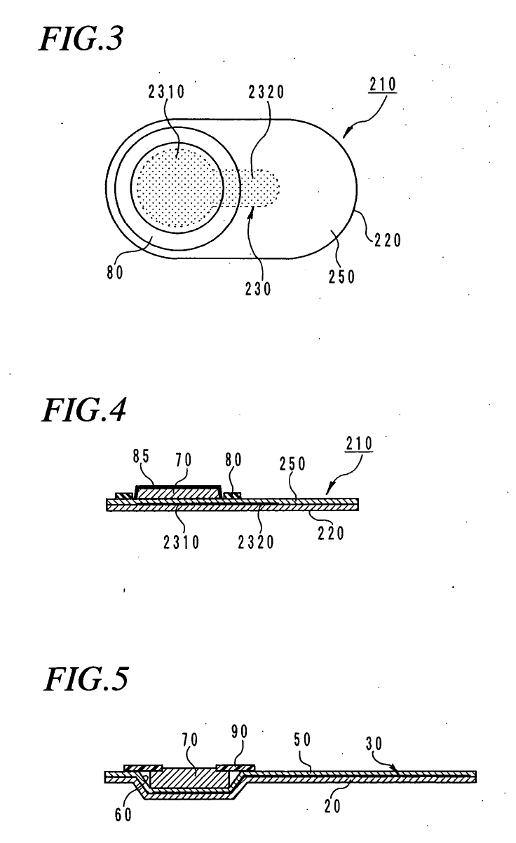

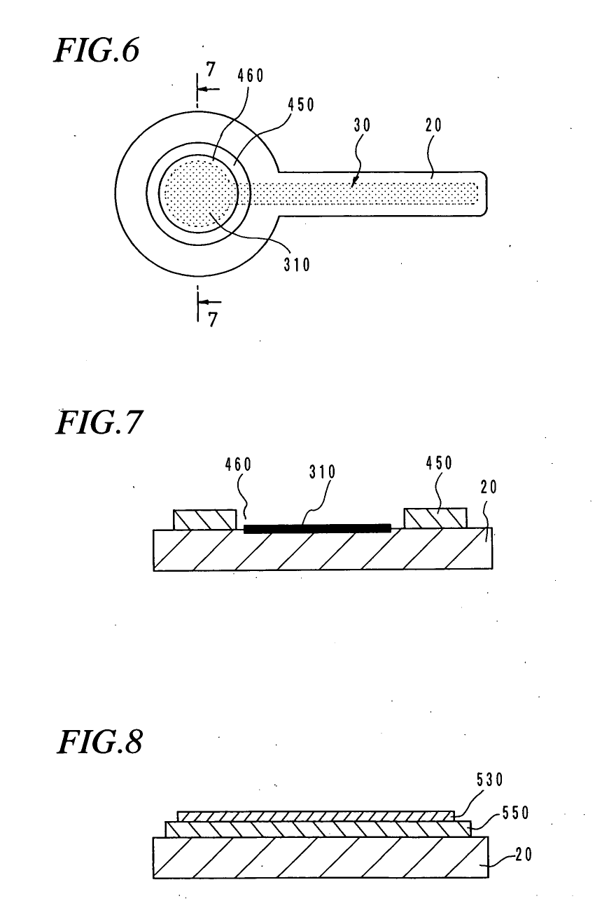

The second embodiment is a flat sheet type electrode device. FIG. 3 shows its plan view, and FIG. 4 shows its sectional view. In this electrode device 210, a polyethylene terephthalate film having a thickness of about 50 to 75 μm is used as a base film 220. An electrode layer 230 laid on one surface of the base film 220 includes a main body part 2310 and a lead part 2320 as in the case with the electrode layer 30 of the first embodiment. The lead part 2320 is comparatively short. Also in this embodiment, a nonwoven fabric 250 is laminated on the base film 220 including the electrode layer 30 as in the case with the first embodiment. A screen printing is made in such a manner as to surround the outer periphery of a circular main body part using an insulative ink, and the painted part is dried at 100 degrees C. for two minutes, so that a ring-shaped insulative layer 80 is formed. As ink for the insulative layer 80, the generally available insulative ink having , for example, the following composition can be used.

| | |
|---|---|
| Polyester | 300 parts |
| (manufactured by Toyo Boseki K.K., merchandise name | |
| “VYRON RV200”) | |
| Bentonite | 24 parts |
| Silica | 9 parts |
| Cyclohexanone | 350 parts |
| Propylene glycol methyl ether acetate | 350 parts |

In the electrode device 210 of the second embodiment, the gel 70 is put into the cup 85 made of polyethylene terephthalate and the inner side of the insulative layer 80 is covered with the cup 85. Then, at the time of use, the cup 85 is removed so that the gel 70 can be intimately contacted with the skin. Also in this second embodiment, the gel 70 is retained with an appropriate retaining force by the nonwoven fabric 250 integral with the base film 220.

The insulative layer 80 of the nonwoven fabric 250 effectively electrically insulates the electrode layer 230 from the skin. Moreover, the insulative layer 80 functions as a bank or dike for the gel 70, so that the gel 70 is protected from flowing out. It is also accepted that the insulative layer 80 is formed in such a manner as to directly contact the electrode layer 230, and the nonwoven fabric 250 is laminated thereon.

### THIRD EMBODIMENT

The third embodiment is, in a sense, a modified embodiment of the first embodiment. In this embodiment, a supporting member 90 is additionally provided to the electrode device of the first embodiment. The supporting member 90 has a ring-like shape. The inside diameter of the ring is set to be smaller than the inside diameter of the concave part 60. Owing to this arrangement, when the gel 70 is charged into the concave part 60, the supporting member 90 can reliably support the gel 70 in the concave part 60 by supporting the peripheral edge part of the gel 70. The supporting member 90 can easily be made by laminating a thermally adhesive resin through one of a suitable lamination methods such as drying, extruding, wetting and the like and then punching out the same. The method for providing this supporting member 90 is useful alone as a method for supporting the peripheral edge part of the gel 70. However, by using this method in combination with the method for supporting the nonwoven fabric 50 through the bottom part of the gel 70, the gel 70 can surely be prevented from falling down.

### FOURTH EMBODIMENT

The fourth embodiment is one mode in which the concave part 60 of the first embodiment is eliminated. Moreover, this fourth embodiment is one example in which a sheet member 450 composed of a nonwoven fabric is selectively provided on the base film 20. The selective sheet member 450 has a ring-like shape and surrounds the outside of a circular main body part 310 of the electrode layer 30 on the base film 20. Also in this embodiment, the sheet member 450 is thick compared with the electrode layer 30, and a concave space 460 for receiving a gel containing a drug is defined at an upper part of the main body part 310 of the electrode layer 30. Accordingly, the gel received in the concave space 460 permeates into the sheet member 450 around the concave space 460 and is thereby retained on the base film 20 side. Since the mode of this fourth embodiment (the mode in which the base film 20 itself is not provided with the concave part) does not require any machining for forming the concave part in the base film, an inexpensive electrode device can be provided. In order to make the electrode device more inexpensive, the base film 20 may be composed of a single layer plastic film. The ring-like sheet member 450 may be formed into a closed ring fully surrounding the circumference of the sheet member 450. It is also an interesting alternative that the sheet member 450 is provided with at least one gap such as a cutout or slit in the peripheral direction as long as there is no worry of an extremely large amount of leakage of the gel. According to the former, the gel can effectively be prevented from leaking out of the concave space 460. According to the latter, it can be expected that a part of the gel enters such a gap or gaps to thereby enhancing the retaining force of the sheet member 450 with respect to the gel.

### FIFTH EMBODIMENT

In the fifth embodiment, a sheet member 550 composed of a nonwoven fabric is laminated on one surface of the base film 20, and an electrode layer 530 is formed on the sheet member 550. The electrode layer 530 consists of a conductive paste ink containing conductive fine particles. Since the electrode layer 530 has such characteristics as to allow the gel to permeate therein, the gel loaded on the electrode layer 530 permeates into the electrode layer 530 and further permeates into the sheet member 550 which forms a lower layer under the electrode layer 530. As a result, the gel is retained on the base film 20 side in a stable manner compared with a case where the sheet member 550 is eliminated. The sheet member 550 may selectively be provided only at the region part where the gel is to be disposed or it may be provided over the entire area of the above-mentioned one surface of the base film 20. In case of the former where the sheet member 550 is selectively provided, some effort should be made such as making the sheet member 550 as much as thinner in order to prevent a possible occurrence of cutoff of the electrode layer 530 at a step part of the sheet member 550. In case of the latter where the sheet member 550 is provided over the entire area, the electrode layer 530 can elastically be supported by elasticity of the sheet member 550.

1. An electrode device for iontophoresis including a base film, an electrode layer being laid on one surface of said base film and a region where a gel containing a drug to be introduced into the body of a patient is disposed being laid on said electrode layer, a voltage being impressed on said gel through said electrode layer to induce ion migration of the drug,
wherein an additional sheet member is integrally laminated on said base film which includes said electrode layer, said sheet member having a property for allowing said gel to permeate therein, so that said gel disposed on said sheet member can be retained with at least a part of said gel permeated in said sheet member.

2. An electrode device for iontophoresis according to claim 1, wherein said sheet member laminated on said base film has a ring-like shape, having a concave space defined at an inner peripheral part thereof, said concave space being adapted to receive said gel therein.

3. An electrode device for iontophoresis according to claim 2, wherein said base film is composed of a plastic film of a single layer.

4. An electrode device for iontophoresis according to claim 1, wherein said base film is composed of a member obtained by laminating a plastic film and a metal film, said laminated member being easily bendable by hand and the bended state being retainable.

5. An electrode device for iontophoresis according to claim 1, wherein said electrode layer includes a main body part corresponding to said area where said gel containing a drug is disposed and a lead part extending from said main body part, said electrode layer further including an insulative layer surrounding said main body part and laid above said lead part in such a manner as to traverse said lead part.

6. An electrode device for iontophoresis according to claim 1, wherein said electrode layer is sandwiched between said base film and said sheet member.

7. An electrode device for iontophoresis according to claim 1, wherein said region of said base film where said gel containing a drug is disposed is concaved.

8. An electrode device for iontophoresis according to claim 7, wherein said base film further includes a support member disposed at a peripheral edge part of said concave part and for supporting said gel receiving in said concave part.

9. An electrode device for iontophoresis according to claim 1, wherein said electrode layer includes a main body part corresponding to said area where said gel containing a drug is disposed and a lead part extending from said main body part, said sheet member having such a configuration as to surround said main body part.

10. An electrode device for iontophoresis including a base film, a region where a gel containing a drug to be introduced into the body of a patient is disposed being laid on one surface of said base film, a voltage being impressed on said gel to induce ion migration of the drug,
wherein said base film is provided at least at said region part where said gel is disposed with a sheet member integrally laminated on said base film and an electrode layer laminated on said sheet member, said sheet member having a property for allowing said gel to permeate therein, so that said gel disposed on said sheet member can be retained with at least a part of said gel permeated in said sheet member.
